(19) United States
(12) Patent Application Publication
(10) Pub. No.: US 2026/0273142 A1
(43) Pub. Date: Sep. 17, 2026

(54) METHODS OF FORMING SHEARED LUBRICIOUS COATED MEDICAL DEVICES

(71) Applicant: Boston Scientific Scimed, Inc., Maple Grove, MN (US)

(72) Inventors: Michael Thomas Frye, Oakdale, MN (US); Justin Theodore Nelson, St. Paul, MN (US); Paul Michael Tomsche, II, Brooklyn Park, MN (US); Sydney Jean Wallace, Lake Elmo, MN (US); Joseph Thomas Delaney, JR., Minneapolis, MN (US); Rick William Dorn, Plymouth, MN (US); Liming Crandall, Lino Lakes, MN (US); Gene Thomas Storbeck, Millis, MA (US); Cole Joseph Christenson, North Oaks, MN (US); Lisa Powell, Rosemount, MN (US)

(73) Assignee: Boston Scientific Scimed, Inc., Maple Grove, MN (US)

(21) Appl. No.: 19/565,936

(22) Filed: Mar. 13, 2026

Related U.S. Application Data
(60) Provisional application No. 63/771,206, filed on Mar. 13, 2025.

(51) Int. Cl. A61L 29/08; A61L 31/04

(52) U.S. Cl. CPC A61L 29/085; A61L 31/048; A61L 2420/02

(57) ABSTRACT

In one aspect, the present disclosure pertains to a method of forming a coated medical device, the method comprising: forming a lubricious coating solution or suspension, wherein the lubricious coating solution or suspension comprises a lubricious polyethylene polymer and a solvent; applying the lubricious coating solution to a substrate to form a coated medical device; and applying a shear stress to the coated medical device to form a sheared lubricious coated medical device.

### CROSS-REFERENCE TO RELATED APPLICATIONS

This application claims the benefit of priority under 35 U.S.C. § 119 of U.S. Provisional Application No. 63/771,206, filed Mar. 13, 2025, the entire disclosure of which is hereby incorporated by reference.

### TECHNICAL FIELD

The disclosure relates to sheared lubricious coatings on medical devices. More particularly, the disclosure is directed to methods of forming and shearing lubricious coatings on medical devices and the resultant sheared lubricious coated medical devices.

### BACKGROUND

A wide variety of intracorporeal medical devices have been developed for medical use and more specifically for surgical and/or intravascular use. Some of these devices include guidewires, catheters, medical device delivery systems (e.g., for stents, grafts, replacement valves, etc.), and the like. These devices are manufactured by any one of a variety of different manufacturing methods and may be used according to any one of a variety of methods. There is an ongoing need to provide alternative medical devices as well as alternative methods for manufacturing and/or using medical devices. For example, as minimally invasive surgical techniques have improved, it has become increasingly common to insert and retrieve medical devices through catheters and the like having considerable length. Accordingly, it is desirable to minimize friction between the catheters that carry such devices and the devices themselves as well as with tissue with which they may come in contact. In the past, the industry has employed various hydrophobic oils and coatings such as olive oil, silicone, and the like as lubricants. One traditional material that has been commonly used to provide a low friction surface is polytetrafluoroethylene (PTFE). Hydrophilic coatings, particularly hydrogels, also have been employed to impart lubricity to a variety of medical devices.

### SUMMARY

In an aspect, a method of forming a coated medical device is provided. The method comprising forming a lubricious coating solution or suspension, wherein the lubricious coating solution or suspension comprises a lubricious polyethylene polymer and a solvent; applying the lubricious coating solution or suspension to a substrate to form a coated medical device; and applying a shear stress to the coated medical device to form a sheared lubricious coated medical device.

In some aspects, which may be used in combination with one or more aspects herein, further comprising applying the lubricious coating solution or suspension to an exterior surface of the substrate and subsequently applying the shear stress along an exterior surface of the coated medical device.

In some aspects, which may be used in combination with one or more aspects herein, wherein the substrate comprises a wire, tube, or mandrel.

In some aspects, which may be used in combination with one or more aspects herein, wherein the shear stress is applied around an entire circumference of the wire, tube, or mandrel.

In some aspects, which may be used in combination with one or more aspects herein, wherein applying the shear stress comprises contacting the exterior surface with a force in a range from about 50,000 to about 600,000 newtons per square meter (N/m²).

In some aspects, which may be used in combination with one or more aspects herein, wherein applying the shear stress comprises contacting the exterior surface with a polymeric member.

In some aspects, which may be used in combination with one or more aspects herein, wherein the polymeric member further comprises a polymeric ring or polymeric pad.

In some aspects, which may be used in combination with one or more aspects herein, wherein the polymeric member is a silicone polymeric member.

In some aspects, which may be used in combination with one or more aspects herein, further comprising applying the shear stress by contacting the exterior surface with the polymeric member with a plurality of passes of the polymeric member that is in a range from 2 passes to 30 passes.

In some aspects, which may be used in combination with one or more aspects herein, wherein applying the lubricious coating is carried out by a coating method selected from a group including spraying, dipping, brushing, or extruding.

In some aspects, which may be used in combination with one or more aspects herein, further comprising applying the shear stress subsequent to drying the coated medical article.

In some aspects, which may be used in combination with one or more aspects herein, further comprising applying the shear stress at ambient conditions.

In some aspects, which may be used in combination with one or more aspects herein, wherein the sheared lubricious coated medical device exhibits an improved lubricity and durability performance as compared to a lubricity and durability performance of the coated medical device.

In some aspects, which may be used in combination with one or more aspects herein, wherein the lubricious polyethylene polymer has a molecular weight in a range from about 500,000 Daltons to about 11,000,000 Daltons.

In some aspects, which may be used in combination with one or more aspects herein, wherein the lubricious polyethylene polymer is an ultra-high molecular weight polyethylene (UHMWPE) having a weight average molecular weight of greater than about 4,000,000 Daltons.

In some aspects, a method of forming a coated medical device is provided. The method comprising forming a lubricious coating solution or suspension, wherein the lubricious coating suspension comprises an ultra-high molecular weight polyethylene (UHMWPE) and solvent, wherein the lubricious coating solution or suspension is per- and polyfluoroalkyl substance (PFAS)-free; applying the lubricious coating solution to a substrate to form a coated medical device; and applying a shear stress to the coated medical device to form a sheared lubricious coated medical device by contacting an exterior surface of the coated medical article with a plurality of passes of a polymeric member.

In some aspects, which may be used in combination with one or more aspects herein, wherein: the coated medical device has an exterior surface that exhibits substantially zero directionality; and the sheared lubricious coated medical device has an exterior surface that exhibits a substantially longitudinal directionality.

In some aspects, a coated medical device is provided. The medical device comprising: a substrate; and a sheared lubricious coating disposed on the substrate, the sheared lubricious coating comprising a lubricious polyethylene polymer.

In some aspects, which may be used in combination with one or more aspects herein, wherein the coated medical device exhibits: initial lubricity and durability performance that is better than a durability of a non-sheared UHMWPE coating disposed on the substrate; and continued lubricity and durability performance that is substantially equal to or better than the non-sheared UHMWPE coating disposed on the substrate.

In some aspects, which may be used in combination with one or more aspects herein, wherein an exterior surface of the coated medical device exhibits: a substantially longitudinal directionality; and a reduced surface roughness as compared to a surface roughness of the coated medical device.

The above summary of some example embodiments is not intended to describe each disclosed embodiment or every implementation of the invention.

### BRIEF DESCRIPTION OF THE DRAWINGS

The disclosure may be more completely understood in consideration of the following detailed description of various embodiments in conjunction with the accompanying drawings, in which:

FIG. 1 illustrates the lubricity and durability performance of various coating compositions.

FIG. 2A illustrates the surface roughness and directionality of a comparative (unsheared) coating composition.

FIG. 2B illustrates the surface roughness and directionality of an example (sheared) coating composition.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). For instance, the term “about” may include values that are within 1 percent, 2 percent, 5 percent, 10 percent of a recited value. In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/of” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

It is generally known to provide substrates, for medical devices or parts of such devices, with a coating for the purpose of reducing the friction between medical devices themselves or between a medical device and a tissue when the device is introduced in an organic body, or within another medical device. Such coatings have also been referred to as lubricious or “slippery” coatings. Catheters and other medical devices used for introduction in blood vessels, urethra, body conduits and the like and guide wires used with such devices are examples of articles which may be provided with coatings. Catheters for balloon angioplasty and biopsy are specific examples of such catheters. Other illustrative medical devices may include, but are not limited to, stents, embolic filters, implantable devices, treatment devices, diagnostic devices, guide catheters, sheaths, etc.

One traditional material that has been commonly used to provide a low friction surface is polytetrafluoroethylene (PTFE). Because of its very low coefficient of friction, it is difficult to wet out the surface of PTFE. Consequently, it can be difficult to adhere other polymers to PTFE, making it difficult to use as a low friction coating. Furthermore, PTFE is very difficult to process. Additionally, use of PTFE along with other per- and polyfluoroalkyl substances (PFAS) may increasingly be subject to various regulatory considerations, environmental concerns, and/or may otherwise be costly. As such, some other approaches seek a lubricious coating that provides similar or improved performance (e.g., lubricity and durability) to traditional coatings such as those that employ PTFE, but is PTFE-free (e.g., does not include any PTFE).

For instance, another material that may provide a low friction surface is ultra-high molecular weight polyethylene (UHMWPE). UHMWPE is a polymer of interest due to its properties including having similar lubricity as polytetrafluoroethylene (PTFE), while offering significantly better wear resistance. The low friction coefficient and high wear resistance of UHMWPE make it suitable for some medical applications such as those that utilize solid UHMWPE implants. Moreover, UHMWPE is not subject to the environmental and/or regulatory considerations associated with PTFE.

Increased lubricity and durability performance is sought for some applications such as various medical device applications (e.g., in which the resultant medical devices are configured to be placed at least temporarily in vivo). As such, the present disclosure pertains to methods and devices with polymeric (e.g., UHMWPE) coatings that have improved lubricity and durability performance. The improved lubricity and durability performance is attributable to applying a shear stress in the coating of a coated substrate (e.g., a wire, tube, or mandrel). The improved performance (e.g., at least improved lubricity and durability performance) of the sheared coated medical devices herein can yield various benefits such as easing the deliverability, placement, and/or removal of the sheared coated medical devices, among other benefits. Moreover, the sheared lubricous medical devices herein are formed without PTFE, but can exhibit comparable or even improved performance characteristics as compared to devices that utilize PTFE coatings which, as detailed herein, present several drawbacks.

At least some aspects of the methods herein can be employed at ambient conditions (e.g., ambient temperature and ambient pressure) and can yield coated medical devices with an absence of PTFE. Hence, the approaches herein can yield various advantages at least in terms of environmental considerations and/or safety and processing as compared to other approaches such as those that utilize PTFE. Yet, the sheared coated medical devices formed via the approaches herein demonstrate similar or improved lubricity and durability performance to coated medical devices produced with PTFE.

The disclosure relates to forming a lubricious coating solution or suspension, applying the lubricious coating solution or suspension to a substrate, and applying a shear stress to the coated substrate. As used herein, the term “solution” refers to a substantially homogeneous mixture in which one or more substances such as UHMWPE (referred to as solutes) are substantially uniformly dispersed at the molecular or ionic level within another substance (referred to as the solvent). The resulting homogeneous mixture is clear or translucent and does not exhibit phase separation at the conditions described herein. As used herein, the term “suspension” refers to a heterogeneous mixture in which solid particles such as UHMWPE are dispersed throughout a liquid. The resulting heterogeneous mixture exhibits phase separation at the conditions described herein. The dispersed UHMWPE particles are not dissolved, may experience swelling, and may settle over time if the heterogeneous mixture is left undisturbed at the conditions described herein. In some embodiments, the lubricious coating solutions or suspensions herein may comprise a lubricious polyethylene polymer (e.g., an ultra-high molecular weight polyethylene (UHMWPE)), a solvent such as a polar or non-polar solvent, and, optionally, a surfactant such as a non-ionic surfactant.

The lubricious coating solutions or suspensions herein can be applied in a variety of manners (e.g., dip coating, spray coating, etc.) to form lubricious coatings having desirable properties. The resultant lubricious coated medical devices (e.g., unsheared lubricious coated medical devices) can exhibit suitable durability and lubricity for some applications, even with the absence of PTFE in the lubricous coated medical devices. However, it has been discovered that the lubricity and durability performance of the unsheared lubricious coated medical devices can be improved by applying a shear stress (e.g., a longitudinal shear) to the coating on the lubricious coated medical device. That is, the lubricious coated medical devices can be sheared, as described herein, to form sheared lubricious coated medical devices that have improved lubricity and durability performance compared to the unsheared lubricious coated medical device. Thus, unlike other coatings, the sheared lubricious coated medical devices herein exhibit a surprising and unexpected increase in lubricity and durability performance, as detailed herein. While various embodiments herein reference thin UHMWPE based coatings for forming coated medical devices, the approaches herein are also amenable to forming thin UHMWPE based liners suitable for forming lined medical devices, among other types of applications (e.g., heat shrink applications, etc.).

Examples of lubricious polyethylene materials useful in the present disclosure include high density polyethylene (HDPE) and ultra-high molecular weight polyethylene (UHMWPE). In some embodiments, the lubricious polyethylene polymer can have a weight average molecular weight from about 500,000 Daltons to about 11,000,000 Daltons. All individual values and sub-ranges from about 500,000 Daltons to about 11,000,000 Daltons are included. For instance, in some embodiments the lubricious polyethylene polymer can have a weight average molecular weight in a range having a lower value of about 500,000 Daltons, about 1,000,000 Daltons, about 2,000,000 Daltons, or about 3,000,000 Daltons and an upper value of about 11,000,000 Daltons, about 10,000,000 Daltons, about 9,000,000 Daltons, about 8,000,000 Daltons, about 7,000,000 Daltons, about 6,000,000 Daltons, about 5,000,000 Daltons, or about 4,000,000 Daltons, among other possibilities. Without wishing to be bound by theory, it is believed that employing a lubricious polyethylene polymer with a higher weight average molecular weight may promote aspects herein. For instance, in some embodiments the lubricious polyethylene polymer can have a weight average molecular weight that is greater than about 4,000,000 Daltons, greater than about 5,000,000 Daltons, greater than about 6,000,000 Daltons, greater than about 7,000,000 Daltons, greater than about 8,000,000 Daltons, greater than about 9,000,000 Daltons, or greater than about 10,000,000 Daltons. In some embodiments, the lubricious polyethylene polymer can have a weight average molecular weight of about 4,000,000 Daltons.

In some embodiments, the lubricious polyethylene polymer can include a UHMWPE. In some embodiments, the lubricious polyethylene polymer can consist essentially of UHMWPE. In some embodiments, the lubricious polyethylene polymer can consist of UHMWPE. In some embodiments, the lubricious polyethylene polymer can consist essentially of UHMWPE and a solvent such as the solvents described herein. In some embodiments, the lubricious polyethylene polymer can consist of UHMWPE and a solvent such as the solvents described herein. In such embodiments, a coating solution or coating suspension including the lubricious polyethylene polymer and solvent may additionally include a relatively small portion of other components (e.g., less than about 5 weight percent of other components e.g., a surfactant such as a non-ionic surfactant based on a total weight of a composition that predominantly includes the UHMWPE and the solvent).

In some embodiments, the lubricious polyethylene polymer such as UHMWPE can be present in a range from about 5 weight percent to about 50 based on a total weight of the lubricious coating solution or suspension. All individual values and sub-ranges from about 5 weight percent to about 50 weight percent are included. For example, the lubricious polyethylene polymer can be present in a range from about 5 weight percent to about 50 weight percent, from about 10 weight percent to about 50 weight percent, from about 15 weight percent to about 50 weight percent, from about 35 weight percent to about 50 weight percent, from about 40 weight percent to about 50 weight percent, from about 45 weight percent to about 50 weight percent, from about 48 weight percent to about 50 weight percent, or from about 49 weight percent to about 50 weight percent based on a total weight of the lubricious coating solution or suspension. For instance, the lubricious polyethylene polymer can comprise about 50 weight percent, or about 49 weight percent based on a total weight of the lubricious coating solution or suspension.

As mentioned, the lubricious coating solutions or suspensions herein can include a polar solvent. Examples of suitable polar solvents include water, ethanol, methanol, isopropyl alcohol, acetone, dimethyl sulfoxide, glycerol, propylene glycol, or any combination thereof. Hence, in some embodiments the polar solvent is selected from a group including water, ethanol, methanol, isopropyl alcohol, acetone, dimethyl sulfoxide, glycerol, propylene glycol, or any combination thereof. In some embodiments, the polar solvent can be selected from a group including water, ethanol, methanol, and isopropyl alcohol, along with any combination thereof. In some embodiments, the polar solvent can include water. In some embodiments, the polar solvent can consist essentially of water (e.g., may include trace amounts of other polar solvents but not any other component such as non-polar solvents). In some embodiments, the polar solvent can include only water (e.g., does not include any other solvent). Employing at least water as the polar solvent can promote aspects herein, e.g., as water is highly polar, biocompatible, and readily permits formation of a suspension with UHMWPE. However, in some embodiments the polar solvent can include ethanol. In some embodiments, the polar solvent can include methanol. In some embodiments, the polar solvent can include isopropyl alcohol. In some embodiments, the polar solvent can include acetone. In some embodiments, the polar solvent can include dimethyl sulfoxide. In some embodiments, the polar solvent can include glycerol. In some embodiments, the polar solvent can be a solution including water and one or more polar solvents that are miscible in water. For instance, such a solution can include water along with one or more of ethanol, methanol, isopropyl alcohol, acetone, dimethyl sulfoxide, glycerol, propylene glycol.

In some embodiments, the solvent is present in a range from about 50 weight percent to about 95 weight percent based on a total weight of the lubricious coating solution or suspension. All individual values and subranges from about 50 weight percent to about 95 weight percent are included. For example, the solvent can be present in a range from about 50 to about 95 weight percent, from about 55 to about 95 weight percent, from about 60 weight percent to about 95 weight percent, or from about 70 weight percent to about 95 weight percent. For instance, the solvent can be about 95 weight percent, about 90 weight percent, about 80 weight percent, about 75 weight percent, about 65 weight percent, about 55 weight percent, or about 50 weight percent based on a total weight of the lubricious coating solution or suspension.

As mentioned, the lubricious coating solution or suspension can include a surfactant. In some embodiments, the surfactant can be an ionic surfactant or non-ionic surfactant. The surfactant such as an ionic surfactant or a non-ionic surfactant can be a liquid at ambient conditions (e.g., at approximately 25 degrees Celsius). In some embodiments, the non-ionic surfactant can be a polyfunctional non-ionic surfactant having both hydrophilic and hydrophobic ends. For instance, the non-ionic surfactant can be liquid at room temperature and can be a bifunctional e.g., having both hydrophilic and hydrophobic ends. In some embodiments, the non-ionic surfactant can be a polysorbate based non-ionic surfactant, an ethoxylated octylphenol based non-ionic surfactant, or a combination of polysorbate based non-ionic surfactant, an ethoxylated octylphenol based non-ionic surfactant. In some embodiments, the non-ionic surfactant can consist essentially of polysorbate based non-ionic surfactant (e.g., may include trace amounts of other non-ionic surfactants but does not include any other components). In some embodiments, the non-ionic surfactant can consist essentially of an ethoxylated octylphenol based non-ionic surfactant (e.g., may include trace amounts of other non-ionic surfactants but does not include any other components). In some embodiments, the non-ionic surfactant can consist of polysorbate based non-ionic surfactant. In some embodiments, the non-ionic surfactant can consist essentially of an ethoxylated octylphenol based non-ionic surfactant.

Examples of suitable polysorbate based non-ionic surfactants include those available under the tradename TWEEN™; available from Croda International plc including TWEEN™ 20 (polysorbate 20) which is derived from lauric acid, TWEEN™ 21 (polysorbate 21) which is derived from myristic acid, TWEEN™ 40 (polysorbate 40) which is derived from palmitic acid, TWEEN™ 60 (polysorbate 60) which is derived from steric acid, TWEEN™ 61 (polysorbate 61) which is derived from steric acid, TWEEN™ 65 (polysorbate 65) which is derived from steric acid, TWEEN™ 80 (polysorbate 80) which is derived from oleic acid, TWEEN™ 81 (polysorbate 81) which is derived from oleic acid, and TWEEN™ 85 (polysorbate 85) which is derived from trioleate. For example, TWEEN™ 20 includes a hydrophilic group manifested as a polyethylene glycol (PEG) chain, which contains approximately 20 ethylene oxide (EO) units, that is polar and thus can form hydrogen bonds with polar solvents such as water. TWEEN™ 20 additionally includes a hydrophobic group manifested as a sorbitan structure (sorbitan monolaurate) that is nonpolar and hydrophobic and thus permits attachment to a lubricious polyethylene polymer UHMWPE.

Examples of suitable ethoxylated octylphenol based non-ionic surfactants include TRITON™ X-100 (available from The DOW Chemical Company) which includes octylphenol ethoxylate, TRITION™ X-100V which includes octylphenol ethoxylate, TRITION™ X-45 which includes octylphenol ethoxylate, and TRITION™ X-114 which includes octylphenol ethoxylate. For example, TRITON™ X-100 includes a hydrophilic group manifested as a polyethylene glycol (PEG) chain, which contains approximately 9-10 ethylene oxide (EO) units, that is polar and capable of forming hydrogen bonds with polar solvents such as water. TRITON™ X-100 additionally includes a hydrophobic group manifested as the octylphenol structure, e.g., that includes an octyl group (C₈H₁₇) that is nonpolar and hydrophobic and thus permits attachment to a lubricious polyethylene polymer UHMWPE. TRITON™ X-45 is based on octylphenol ethoxylate, with a hydrophilic (water-attracting) ethylene oxide chain where the “45” in the name indicates the average degree of ethoxylation.

In some embodiments, the surfactant such as a non-ionic surfactant is present in a range from about 0.1 weight percent to about 5.0 weight percent based on a total weight of the lubricious coating solution or suspension. Any individual values and sub-ranges from about 0.1 weight percent to about 5.0 weight percent are included. For example, the surfactant is present in a range from about 0.1 weight percent to about 4.0 weight percent, 0.5 weight percent to about 4.0 weight percent, from about 0.5 weight percent to about 3.5 weight percent, from about 0.5 weight percent to about 3.0 weight percent, from about 0.5 weight percent to about 2.5 weight percent, from about 1.0 weight percent to about 2.5 weight percent, from about 1.0 weight percent to about 2.0 weight percent, or from about 0.5 weight percent to about 1.5 weight percent based on a total weight of the lubricious coating solution or suspension. For instance, the non-ionic surfactant can be about 0.1 weight percent, about 0.5 weight percent, about 1.0 weight percent, from about 1.5 weight percent, or about 2.0 weight percent based on a total weight of the lubricious coating solution or suspension.

In some embodiments, a ratio of the lubricious polyethylene polymer (e.g., UHMWPE) to the solvent in the lubricous coating solution or suspension can be about 1:1. In some embodiments, the lubricious polyethylene polymer and the solvent can together form at least 70 weight percent, at least 80 weight percent, at least 90 weight percent, at least 95 weight percent, at least 96 weight percent, at least 97 weight percent, at least 98 weight percent, at least 99 weight percent, or 100 weight percent of a total weight of the lubricious coating solution or suspension. In such embodiments, a balance of the lubricious coating solution or suspension can be manifested at least in part by the surfactant such as a non-ionic surfactant. In some embodiments the lubricious polyethylene polymer such as a UHMWP and the solvent such as decalin can collectively form the entire weight of a lubricious coating solution. In some embodiments the lubricious polyethylene polymer such as a UHMWPE, the solvent such as water, and a non-ionic surfactant can collectively form the entire weight of a lubricious coating suspension. For instance, in some embodiments, the lubricious polyethylene polymer can be present at about 50 weight percent, the polar solvent can be present at about 49 weight percent, and the surfactant can be present at about 1 weight percent of a total weight of the lubricious coating suspension, among other possible values. However, in some embodiments, the lubricious coating solutions or suspensions herein can include additional components (e.g., additives), as described herein.

In some embodiments, the lubricious coating solution or suspension and/or a pre-solution or pre-suspension mixture can be agitated. For instance, a magnetic stir-bar or other agitation mechanism can be employed to agitate a pre-suspension mixture including the lubricious polyethylene polymer and the solvent, and when present the surfactant such as a non-ionic surfactant thereby forming the lubricious coating solution or suspension. In such embodiments, the resultant lubricious coating solution or suspension can continue to be agitated e.g., to ensure that the lubricious polyethylene remains suspended or emulsified in the lubricious coating solution or suspension.

In some embodiments, the lubricous coating solutions herein can be formed and applied to a substrate at an elevated temperature (e.g., an elevated temperature in a range from about 120 degrees Celsius to about 200 degrees Celsius). However, in some embodiments, the lubricious suspensions herein can be formed and applied to a substrate at ambient conditions.

The lubricious coating solutions and suspensions herein, the resultant lubricious coated medical devices, and the resultant sheared lubricious coated medical devices, as detailed herein, are PFAS-free. For instance, unlike some previous approaches and resultant coated medical devices, the lubricious coating solution or suspension and the resultant coated medical devices and sheared coated medical devices herein are fluorinated ethylene propylene (FEP)-free and PTFE-free. As used herein, being PFAS-free generally refers to including no appreciable or detectable amount of PFAS.

In some embodiments, a hydrophilic polymer may, in addition to the lubricous polymer and a solvent, also be included in the lubricious coating suspensions (e.g., in the absence of a surfactant) herein. Examples of such polymers include polyethylene glycol, polypropylene glycol, polyvinylpryrrolidone, and/or hydrophilic urethane polymers, including acrylated urethanes, for example. The polymer may comprise monomer units from one or more monomers having organic acid functional groups. Examples of such monomers include acrylic acid, methacrylic acid, and/or isocrotonic acid. However, in some embodiments, a hydrophobic polymer may be included in the lubricious coating suspension (e.g., in the presence of a surfactant). That is, the coating suspensions herein can include a hydrophilic polymer such as a hydrophilic UHMWPE or a hydrophobic polymer such as a hydrophobic UHMWPE.

In some embodiments, a free radical initiator may also be included in the lubricious coating solutions or suspensions. The free radical initiator may be, for example, a photoinitiator. Non-limiting examples of free radical photoinitiators that may be employed include benzophenones, ketones, acrylated amine synergists, alpha-amino ketones, acyl phosphine oxides including bis-acyl phosphine oxides, and benzil ketals. More specific examples of photoinitiators suitable for use herein include, but are not limited to, 2-phenyl-1-indanone; IRGACURE 184 from Ciba Specialty Chemicals, BENACURE 184 from Mayzo and SARCURE SR1122 from Sartomer, all of which are 1-hydroxylcyclohexylphenyl ketone (HCPK) initiators; BENACURE BP benzophenone; BENACURE 651 and IRGACURE 651, both of which are benzil dimethyl ketal or 2,2′dimethoxy-2-phenylacetophenone; BENACURE 1732 hydroxy-2-methyl-1-phenyl-1-propanone; IRGACURE 819 bis(2,4,6-trimethylbenzoyl)-phenylphosphineoxide, IRGACURE 907 2-Methyl-1-[4-(methylthio)phenyl]-2-(4-morpholinyl)-1-propanone; IRGACURE 369 morpholinoketone; and so forth and blends thereof. Photoinitiators are also available commercially in a variety of blends. Examples of commercially available blends include, but are not limited to, SARCURE SR1136 that is a blend of 4-methylbenzophenone and benzophenone; SARCURE SR1137 that is a blend of trimethylbenzophenone and methylbenzophenone; and BENACURE 500, that is a blend of 1-hydroxylcyclohexylphenyl ketone and benzophenone.

Other optional additives may be used in the lubricious coating solutions or suspensions of the present disclosure including flow or viscosity modifiers, antioxidants, coupling agents, surfactants, and/or therapeutic agents. Any such additives may be incorporated into the composition at levels of 10 weight percent or less (e.g., ranging from 10 weight percent to 5 weight percent to 2 weight percent to 1 weight percent to 0.5 weight percent or less), based on the dry weight (e.g., excluding solvent) of the composition.

Typically, the lubricious coating solutions or suspensions for use in forming the lubricious coated medical devices herein may contain from about 0.25 percent to about 10 percent solids, about 0.5 percent to about 10 percent solids, about 1 percent to about 10 percent solids, or about 2 percent to about 7 percent solids, or about 3 percent to about 6 percent solids. The solids of the lubricious coating solutions or suspensions may include or may be limited to the lubricious polyethylene polymer.

The lubricious coating solution or suspension may be applied to the medical device by any method known in the art including, but not limited to, spraying, dipping, rolling, painting (e.g., brush painting, sponge painting, etc.), and so forth. In some embodiments, the lubricious coating compositions herein are applied to the surface of a substrate of a medical article as a solution. In some embodiments, the lubricious coating compositions herein are applied to the surface of a substrate of a medical article as a suspension. Once the solution or suspension is applied to the substrate, the coating may then be allowed to dry, by evaporation of the solvent. The solvent may be more readily evaporated at an elevated temperature, although room temperature drying is typically acceptable.

In some embodiments, a shear stress is applied along an exterior surface of the coated medical device. As mentioned, the coated medical device can be formed by applying a lubricous coating solution or suspension to various substrates such as a wire, tube, or mandrel. In some embodiments, the lubricious coating solution or suspension can be applied to an entire exterior surface of the substrate. In some embodiments, the shear stress can be applied to a portion or the entire exterior coated surface of the coated medical device. For instance, the shear stress can be applied around an entire circumference and along an entire longitudinal length of the substrate such as a wire, tube, or mandrel.

The shear stress can be applied by contacting the exterior surface of the coated medical device with a polymeric member. In some embodiments, the polymeric member can have a Shore A hardness in a range from about 50 to 70. For instance, the polymeric member can have a Shore A hardness of about 60. Suitable materials for the polymeric member include those described herein such as silicon. That is, in some embodiments the polymeric member can be silicone polymeric member. The silicone polymeric member can be formed entirely of silicon. The polymeric member can be manifested as a polymeric ring or polymeric pad. The polymeric ring refers to an annulus that is configured to permit the coated medical device to pass longitudinally therethrough to apply the shear stress to a portion of the coated medical device that is in contact with the polymeric ring. An example of a suitable silicone ring is a 70 durometer FDA FKM ring available from FasTest Inc. The polymer pads refer to two or more pads that are collectively configured to contact and apply the shear stress to a portion of the coated medical device that is in contact with polymeric ring.

The shear stress is applied by contacting the exterior surface with the polymeric member one or more times. For instance, in some embodiments, the shear stress can be applied to the coating of the coated medical article with a plurality of passes of the polymeric member (e.g., contacting the same portion of the polymeric member a plurality of times with the polymeric member). In such instances, the plurality of passes is in a range from 2 passes to 30 passes, from 2 to 20 passes, from 2 to 10 passes, or from 2 to 6 passes. Without wishing to be bound by theory it is believed the employing a plurality of passes to apply the shear stress can increase the directionality of the exterior surface of the sheared coated medical devices, improve lubricity or durability performance, and/or mitigate the potential for the coating to delaminate from the substrate (e.g., at least in part to applying less force to the coating on each pass as compared to an amount of force that would be utilized with only one pass to achieve the same or similar mechanical changes in the exterior surface.)

In some embodiments, the shear stress can be applied at a pressure (e.g., the polymeric member is configured to exert on the coating of the coated medical device by the polymeric member) that is in a range from about 10,000 newtons/meter² (N/m²) to about 600,000 N/m² or about 50,000 to about 600,000 N/m². The force can be exerted on the coating of the coated medical device in a substantially longitudinal direction. For instance, in some embodiments the exterior surface of the coated medical device can be contacted with a longitudinal force in a range from about 50,000 to about 600,000 newtons per square meter (N/m²) to apply the shear stress to the coated medical device. For example, the exterior surface of the coated medical device can be contacted with a longitudinal force of about 50,000 N/m². However, in some embodiments the polymeric member can exert additional forces (e.g., a compressive force in a direction that is normal to longitudinal axis of the coated medical device) on the coating of the coated medical device. In embodiments that employ a plurality of passes to apply the shear, each of the passes can apply the shear stress to the coating of the coated medical device with the same amount of force. However, in some embodiments the force associated with a given (e.g., first) pass can be different than the force associated with a subsequent (e.g., second) pass.

In some embodiments, the shear stress can be applied to the coated medical article subsequent to drying the coated medical article, as detailed herein. In some embodiments, the shear stress can be applied at the ambient conditions such as ambient temperature and ambient pressure.

As detailed herein, the sheared coated medical devices can exhibit an improved lubricity and durability performance as compared to a lubricity and durability performance of the coated medical device. The improvement can correspond to at least a 20 percent, at least a 30 percent, at least a 40 percent or at least a 50 percent improvement in lubricity and durability performance or can correspond to at least a 5 percent, at least a 10 percent, at least a 15 percent, at least a 20 percent, at least 30 percent, at least a 40 percent, or at least a 50 percent improvement. For instance, both an initial lubricity and durability performance and a continued lubricity and durability performance can be improved as compared to the unsheared coated medical devices. In some instances, the sheared coated medical devices can exhibit at least a 30 percent, at least a 40 percent, or at least a 50 percent improvement in initial lubricity and durability performance.

The improved lubricity and durability performance can be at least partially attributable to the presence in a longitudinal directionality in the coatings of the sheared coated medical devices and/or an improved surface roughness. For instance, each of an average surface roughness (Sa), a root mean square roughness (Sq), and a mean roughness depth (Sz) can be improved. The improvement in surface roughness can correspond to at least a 20 percent, at least a 30 percent, at least 40 percent, or at least a 50 percent improvement in surface roughness. The improved surface roughness is at least partially attributable to an improved crystallinity percentage as compared to a crystallinity percentage of the coated medical device and/or an improved orthorhombic crystallite size as compared to an orthorhombic crystallite size of the coated medical device.

As used herein, the presence of the longitudinal directionality in the coating of the sheared lubricious coated medical devices refers to the presence of substantially longitudinally extending shear lines or marks along the coating. For instance, the substantially longitudinally extending shear lines or marks may be manifested as a series of substantially longitudinally extending recesses such as those that are positioned circumferentially about the exterior surface of the sheared lubricious coated medical article. The presence of the substantially longitudinally extending shear lines or marks may be observed in some instances visually with the aid of magnification (e.g., with a microscope). In some embodiments, the substantially longitudinally extending shear lines or marks can have a longitudinal length that is at least about 0.01 millimeters. In some embodiments, the substantially longitudinally extending shear lines or marks can have a width (taken in a direction normal to the longitudinal length) of at least about 0.005 millimeters. In some embodiments, the substantially longitudinally extending shear lines or marks can have a depth (axially extending depth into the exterior surface of the coating) of at least about 0.001 millimeters. In some embodiments, a quantity of the substantially longitudinally extending shear lines or marks can be in a range from about 10 to about 100, about 10 to about 500, about 10 to about 1000. In some embodiments, the substantially longitudinally extending shear lines or marks can extend along an entire longitudinal length of the exterior surface of the sheared lubricious coated medical device. The length, depth, width, and/or a quantity of the substantially longitudinally extending shear lines or marks can vary, for instance, depending on a thickness and/or hardness of a coating, an quantity of shearing passes, an amount of shearing force applied by the polymeric member (e.g., a ring or pads, etc.) and/or a size (e.g., a diameter, circumference, and/or longitudinal length, etc.) of a substrate. In some embodiments, a direction of the shear lines or marks can substantially correspond to a direction of the shear stress applied to the exterior coating of the coated medical device.

In some embodiments, the sheared or unsheared coated medical articles that have been coated with the lubricious coating solution or suspension can undergo post processing. For instance, the sheared or unsheared coated medical device can be post processed by heating the coated medical device in an environment (e.g., an oven) at a temperature in a range from about 135 degrees Celsius and 500 degrees Celsius for a period of time (e.g., about 10 minutes, etc.). Such post processing can enhance a smoothness or lubricity of the coated medical article. For example, without wishing to be bound by theory, such post processing is believed to cause sintering, melting, and/or annealing of the lubricious polyethylene polymer and thereby impart increased uniformity in an exterior surface of the sheared or unsheared coated medical device.

A variety of substrate materials may be used in conjunction with the present disclosure including organic and inorganic substrates, typically polymer substrates, metal substrates and glass substrates, among others. Examples of metal substrates include pure metals such as platinum, gold, iridium and titanium, plated metals (e.g., silver plated copper), or metal alloys such as stainless-steel including platinum enriched stainless steel (PERSS), Nitinol alloys, and cobalt chromium alloys. An example of a suitable metal substrate is MP35N, a nickel cobalt alloy.

Examples of polymer substrates include the following: (a) olefin homopolymers and copolymers, including homopolymers and copolymers of C2-C8 alkenes, for example, polyethylene and polypropylene, ethylene-vinyl acetate copolymers (EVA), and isobutylene-styrene copolymers, including block copolymers comprising one or more polystyrene blocks and one or more polyisobutylene blocks, for instance, poly(styrene-b-isobutylene-b-styrene) (SIBS), among others, (b) polyamides such as nylons, polyether-polyamide block copolymers such as poly(tetramethylene oxide-b-polyamide-12) block copolymer, available from Elf Atochem as PEBAX, among others, (c) fluoropolymers, including homopolymers and copolymers of C2-C8 alkenes in which one or more hydrogen atoms are substituted with fluorine, for example, polytetrafluoroethylene (PTFE), polyhexafluoropropene (PVDF), polyvinylidene fluoride (PVDF), and poly(vinylidene fluoride-co-hexafluoropropene) (PVDF-HFP), among others, (d) polyurethane copolymers, including copolymers that are polyether based, polyester based, polycarbonate based, aromatic based and aliphatic based, including polyisobutylene based polyurethanes (PIB-PU), (e) silicone homopolymers and copolymers (also referred to as polysiloxanes) such as polydimethylsiloxane, and (f) various thermoplastics such as polyether ether ketone (PEEK).

Examples of substrates include medical article substrates, specific examples of which include medical device substrates, for instance, implantable or insertable medical device substrates. A variety of devices may thus be partially or completely coated with compositions in accordance with the present disclosure, including, for example, catheters (e.g., renal or vascular catheters), balloons, catheter shafts, guide wires, filters (e.g., vena cava filters), stents (including coronary vascular stents, cerebral stents, urethral stents, ureteral stents, biliary stents, tracheal stents, gastrointestinal stents and esophageal stents), stent grafts, cerebral aneurysm filler coils (including Guglilmi detachable coils and metal coils), vascular grafts, myocardial plugs, patches, pacemakers and pacemaker leads, heart valves, vascular valves, tissue engineering scaffolds for cartilage, bone, skin and other in vivo tissue regeneration.

Lubricious coating solutions or suspensions in accordance with the present disclosure may be cured, for example, by exposing the coating composition to heat or actinic radiation such as UV light for a short period of time and/or allowing drying (solvent evaporation). In some instances, drying can be performed at an elevated temperature. The amount of time needed to dry or cure the surface is dependent on the source of energy, the relative amounts of constituents in the composition, the thickness of the coating desired, and other factors. Generally, the amount of time required for drying or thermal cure can be less than one minute such as about three seconds or less at an elevated temperature. Curing around and along the substrate can be accomplished by incrementally or continuously using irradiation from multiple angles using spaced lamps and/or reflectors; rotation of the substrate, light source or light beam; longitudinal movement of the substrate, light source or light beam; or a combination of such techniques.

In some embodiments, a thickness of the lubricious coating on the substrate may be in a range from about 2 micrometers to about 300 micrometers or from about 1 micrometer to about 50 micrometers. In some embodiments, a thickness of the lubricious coating on the substrate may be in the range of from 0.1 micrometers or less to 20 micrometers or more (e.g., from 0.1 to 0.2 to 0.5 to 1 to 2 to 5 to 10 to 20 micrometers), or about 0.1 to about 5 micrometers. The lubricious coating thickness will be affected by the percent solids in the coating and the technique of application, among other factors. Multiple coatings may be applied to achieve a desired coating thickness. However, in some embodiments the lubricious coating can be formed from an individual coating (e.g., an individual application of the lubricious coating suspension to the substrate). In some embodiments, a coating thickness of the coated medical device can be substantially equal to a coating thickness of the sheared lubricious coated medical device (e.g., despite the application of the shear force and the resultant mechanical changes in the coating such as the presence of directionality in the sheared coating).

In some embodiments, the lubricious coatings herein can be sterilized. In some embodiments the lubricious coatings described herein may comprise a therapeutic agent, for example, selected from antimicrobial agents, antibiotic agents, anti-cancer agents, anti-oxidizing agents (e.g., Vitamin E), agents for treating calcifications, antirestenotic agents and antithrombotic agents, and combinations thereof. The therapeutic agent(s) may be added to the lubricious coating prior to curing or drying or applied onto the coating after it has been dried, cured, and/or sterilized. In some embodiments, therapeutic agent(s) carried in the lubricious polyethylene polymer-based coating may remain in the coating or elute out of the coating when the coating is wet, thereby delivering the therapeutic agent(s) to immediately adjacent areas of the body.

The invention is illustrated by the following non-limiting examples.

### Examples 1 and Comparative Examples 1-2

The coating thickness for each of the examples was approximately 50.8 micrometers [0.002 inches] or less. Unless otherwise indicated, the materials herein were procured from SIGMA-ALDRICH.

Example 1 and the comparative examples 1-2 were formed as follows.

Comparative Example 1 (CE 1—PFAS Coated wire): A PTFE coated wire available from OLYMPUS AMERICA MEDICAL VISIGLIDE (PES/PTFE) was provided as comparative example 1 (CE 1).

Comparative Example 2 (CE 2—Solution cast coated medical device with an absence of application of a shear stress): A lubricious polyethylene powder (UHMWPE powder with average molecular weight of about 4,200,000 Daltons; tradename GUR2126; available from Celanese) was dissolved in about 1 weight percent decalin at about 130 degrees Celsius under agitation with a magnetic stir bar to form a lubricious coating solution. A nitinol catheter wire (available from FORT WAYNE MATERIALS) was dipped into the lubricious coating solution and allowed to dry at about 150 degrees Celsius for about 1 minute to form a coated medical article. The coated medical device corresponds to comparative example 1 (CE 1). The coated medical device is PFAS-free.

The coating of the coated medical device (e.g., once dried and the solvent has substantially evaporated) can predominantly include UHMWPE (e.g., including greater than or equal to 99 weight percent UHMWPE or 100 weight percent UHMWPE). In some embodiments, trace amounts of the residual polar solvent and/or trace amounts of residual surfactant (if present in the initial lubricious coating solution) may remain in the coating of the coated medical device.

### Example 1 (EX 1—Solution Cast Coated Medical Device with an Application of a Longitudinal Shear Stress)

A PFAS-free solution cast coated medical device was formed and allowed to dry in accordance with CE 2. Subsequent to formation and drying of the coated medical device, a substantially longitudinal shear was applied to an exterior surface of the coated medical device. The shear was applied by contacting the entire exterior surface of the coated medical device with two opposingly orientated silicone pads. The silicone pads had a Shore A surface hardness of approximately 60 and are available from Harland Medical Systems under the part no. 907294. The pads were affixed to an Oak River Technology DL1000 durability & lubricity test system. The coated medical article was passed in a substantially longitudinal manner (e.g., with an absence or substantial absence of any circumferential rotation of the coated medical device) through the silicone pads such that an entire longitudinal length of the coated medical device passed through the silicone pads in substantially uniform longitudinal direction. The silicone pads were configured to contact the exterior surface with a substantially uniform force along the entire longitudinal length of the coated medical device. The force (e.g., compressive force) was about 50,000 newtons/meter² (N/m²). The process of passing the entire length of the coated medical article through the silicone pads was repeated 30 times (e.g., passes) to form the sheared coated lubricious medical device that corresponds to example 1 (EX 1). Each pass was performed with the application of substantially the same amount of force.

CE 1-2 and EX 1 were each tested for lubricity and durability (L&D). To test for L&D, the coated wires of CE 1-2 and EX 1 are secured by a clamping mechanism at a normal force of about 300 grams (g). The force required to pull the coated articles of CE 1-2 and EX 1 through the clamp were measured over a thirty-cycle test. Lower frictional force (grams) indicates improved lubricity.

The lubricity and durability testing were conducted under both dry and wet conditions. As used herein, dry conditions refer to contacting the materials under test with a dry silicone pad in the clamping mechanism. As used herein, wet conditions refer to contacting the materials under test with a silicone pad in the clamping mechanism that is immersed in water.

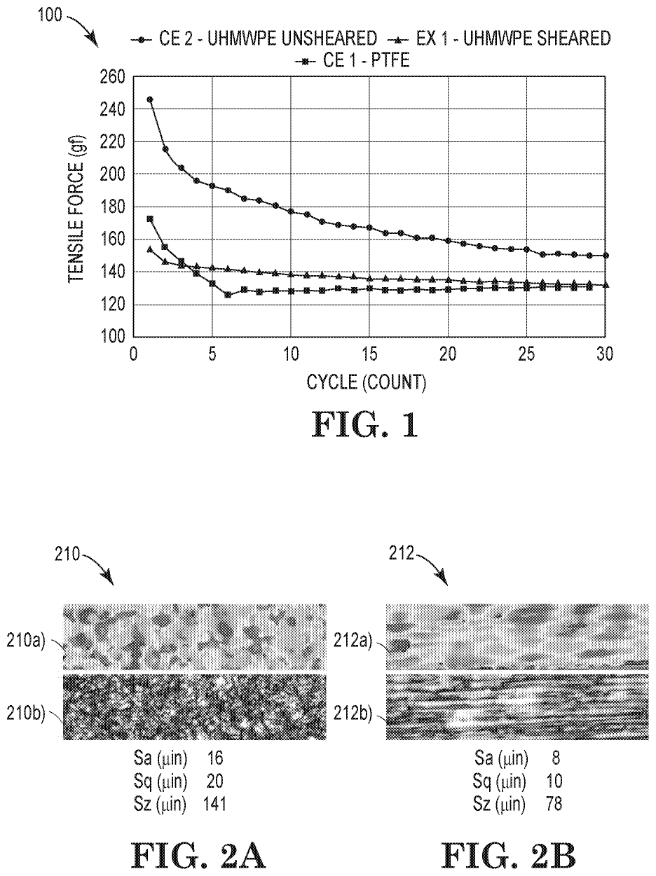

Lubricity and durability performance of the articles of CE 1-2 and EX 1 was determined based on observed frictional forces of the materials undertest after a given quantity of cycles (e.g., after each cycle from 1 to 30 cycles). FIG. 1 illustrates a graph of the average force required (in grams) to pull an example wire through the clamp for each of the coating compositions under dry conditions over a number of cycles (e.g., up to 30 cycles). Table 1 provides the lubricity and durability performance data that is the basis for FIG. 1.

TABLE 1

| | CE 1 - | CE 2 - No | EX 1 - |
|---|---|---|---|
| Cycles | PTFE | shearing | Shearing |
| 1 | 172.171 | 246.006 | 153.557 |
| 2 | 154.864 | 214.838 | 146.197 |
| 3 | 146.820 | 204.131 | 144.015 |
| 4 | 139.007 | 196.436 | 143.186 |
| 5 | 132.65 | 192.429 | 142.375 |
| 6 | 125.762 | 190.173 | 141.507 |
| 7 | 128.913 | 185.008 | 140.460 |
| 8 | 127.987 | 183.578 | 139.909 |
| 9 | 128.147 | 180.669 | 139.022 |
| 10 | 128.350 | 176.790 | 138.266 |
| 11 | 128.280 | 175.143 | 137.51 |
| 12 | 128.291 | 171.113 | 137.507 |
| 13 | 129.971 | 168.883 | 137.197 |
| 14 | 128.808 | 163.402 | 136.529 |
| 15 | 129.996 | 160.824 | 135.789 |
| 16 | 128.967 | 160.637 | 135.587 |
| 17 | 128.664 | 159.115 | 135.901 |
| 18 | 129.185 | 157.545 | 135.493 |
| 19 | 128.965 | 155.860 | 135.091 |
| 20 | 129.477 | 159.115 | 134.808 |
| 21 | 129.732 | 157.454 | 134.307 |
| 22 | 129.746 | 155.860 | 133.974 |
| 23 | 130.323 | 154.635 | 134.079 |
| 24 | 130.397 | 153.825 | 133.610 |
| 25 | 130.161 | 153.752 | 133.332 |
| 26 | 130.721 | 150.790 | 132.844 |
| 27 | 131.014 | 151.017 | 132.417 |
| 28 | 131.037 | 150.701 | 132.207 |
| 29 | 130.807 | 149.989 | 132.116 |
| 30 | N/A | 149.93 | 132.019 |

“N/A” indicates that the corresponding data value is not available.

As can be seen in FIG. 1, the examples that utilized a sheared lubricious coating formed at least of a lubricious polyethylene polymer realized comparable or improved lubricity and durability performance, illustrated as EX 1 in FIG. 1, to the comparative examples CE 1-2. Thus, EX 1 exhibited satisfactory lubricity and durability performance for the entire duration of the lubricity and durability testing (e.g., at each of 1 cycle, 10 cycles, and 30 cycles). Such lubricity and durability performance can be particularly advantageous for the implantable or insertable medical devices herein such as those may encounter fluids during implantation and/or insertion.

Without wishing to be bound by theory, it is believed that the lubricity and durability performance of EX 1 is attributable to the above-mentioned application of a shear stress to the coating of the coated lubricious medical article (e.g., coated medical article). For example, this mechanical shearing of the coating of the coated medical device results in mechanical changes to the surfaces properties of the coating. As a result, the sheared lubricious coated medical devices herein exhibit a substantially longitudinal directionality and have an improved (reduced) surface roughness.

For instance, the respective exterior surfaces of the sheared coated medical device of EX 1 and the exterior surface of the coated medical device of CE 2 were visually observed with a Zygo NewView 8300 optical profiler. FIGS. 2A and 2B correspond to observed images to the exterior surfaces of the sheared coated medical device of EX 1 and the exterior surface of the coated medical device of CE 2 using the microscopes. As illustrated in FIG. 2A, the exterior surface 210a of the coated medical device of CE 2 as observed with the Zygo NewView 8300 optical profiler has an absence of directionality (e.g., does not include substantially longitudinally extending shear lines or marks), whereas the exterior surface 212a of the sheared coated medical device of EX 1 has a directionality (e.g., has the presence of substantially longitudinally extending shear lines or marks). Additionally, the exterior surface 210b of the coated medical device of CE 2 as observed with the Zygo NewView 8300 Optical Profiler has a higher or increased surface roughness (e.g., an average surface roughness (Sa) of about 0.0004 millimeters (mm) [16 micro inches (μin)], a root mean square roughness (Sq) of about 0.0005 mm [20 μin], and a mean roughness depth (Sz) of about 0.0035 mm [141 μin]), whereas the exterior surface 212b of the sheared coated medical device of EX 1 has a lower or decreased surface roughness (e.g., an Sa of about 0.0002 mm [8 μin], a Sq of about 0.0003 mm [11 μin], and a Sz of about 0.0020 mm [78 μin]). The surface roughness values (Sa, Sq, Sz) of the exterior surfaces 210, 212 were obtained by a Zygo NewView 8300 optical profiler.

Although various embodiments are specifically illustrated and described herein, it will be appreciated that modifications and variations of the present disclosure are covered by the above teachings and are within the purview of the appended claims without departing from the spirit and intended scope of the invention.

What is claimed is:

1. A method of forming a coated medical device, the method comprising:
forming a lubricious coating solution or suspension, wherein the lubricious coating solution or suspension comprises a lubricious polyethylene polymer and a solvent;
applying the lubricious coating solution or suspension to a substrate to form a coated medical device; and
applying a shear stress to the coated medical device to form a sheared lubricious coated medical device.

2. The method of claim 1, further comprising applying the lubricious coating solution or suspension to an exterior surface of the substrate and subsequently applying the shear stress in a substantially longitudinal direction along an exterior surface of the coated medical device.

3. The method of claim 2, wherein the substrate comprises a wire, tube, or mandrel.

4. The method of claim 3, wherein the shear stress is applied along in the substantially longitudinal direction around an entire circumference of the wire, tube, or mandrel.

5. The method of claim 4, wherein applying the shear stress comprises contacting the exterior surface with a force in a range from about 50,000 to about 600,000 newtons per square meter (N/m²).

6. The method of claim 1, wherein applying the shear stress comprises contacting the exterior surface with a polymeric member.

7. The method of claim 6, wherein the polymeric member further comprises a polymeric ring or polymeric pad.

8. The method of claim 6, wherein the polymeric member is a silicone polymeric member.

9. The method of claim 6, further comprising applying the shear stress by contacting the exterior surface with the polymeric member with a plurality of passes of the polymeric member that is in a range from 2 passes to 30 passes.

10. The method of claim 1, wherein applying the lubricious coating is carried out by a coating method selected from a group including spraying, dipping, brushing, or extruding.

11. The method of claim 1, further comprising applying the shear stress subsequent to drying the coated medical article.

12. The method of claim 11, further comprising applying the shear stress at ambient conditions.

13. The method of claim 1, wherein the sheared lubricious coated medical device exhibits an improved lubricity and durability performance as compared to a lubricity and durability performance of the coated medical device.

14. The method of claim 1, wherein the lubricious polyethylene polymer has a molecular weight in a range from about 500,000 Daltons to about 11,000,000 Daltons.

15. The method of claim 1, wherein the lubricious polyethylene polymer is an ultra-high molecular weight polyethylene (UHMWPE) having a weight average molecular weight of greater than about 4,000,000 Daltons.

16. A method of forming a coated medical device, the method comprising:
forming a lubricious coating solution or suspension, wherein the lubricious coating suspension comprises an ultra-high molecular weight polyethylene (UHMWPE) and solvent, wherein the lubricious coating solution or suspension is per- and polyfluoroalkyl substance (PFAS)-free;
applying the lubricious coating solution to a substrate to form a coated medical device; and
applying a shear stress to the coated medical device to form a sheared lubricious coated medical device by contacting an exterior surface of the coated medical article with a plurality of passes of a polymeric member.

17. The method of claim 16, wherein:
the coated medical device has an exterior surface that exhibits substantially zero directionality; and
the sheared lubricious coated medical device has an exterior surface that exhibits a substantially longitudinal directionality.

18. A coated medical device comprising:
a substrate; and
a sheared lubricious coating disposed on the substrate, the sheared lubricious coating comprising a lubricious polyethylene polymer.

19. The coated medical device of claim 18, wherein the coated medical device exhibits:
initial lubricity and durability performance that is better than a durability of a non-sheared UHMWPE coating disposed on the substrate; and
continued lubricity and durability performance that is substantially equal to or better than the non-sheared UHMWPE coating disposed on the substrate.

20. The coated medical device of claim 18, wherein an exterior surface of the coated medical device exhibits:
a substantially longitudinal directionality; and
a reduced surface roughness as compared to a surface roughness of the coated medical device.
